(19) United States
(12) Patent Application Publication
(10) Pub. No.: US 2024/0409991 A1
(43) Pub. Date: Dec. 12, 2024

(54) INTEGRATED SYSTEM FOR CHEMICAL, BIOCHEMICAL OR MOLECULAR BIOLOGICAL REACTIONS IN A MICROPLATE

(71) Applicant: BAYER AKTIENGESELLSCHAFT, Leverkusen (DE)

(72) Inventors: Andreas SCHADE, Leverkusen (DE); Mike KÜSTER, Leverkusen (DE); Klaus OCHMANN, Leverkusen (DE); Philipp PFLÜGER, Leverkusen (DE); Robin GESELL, Leverkusen (DE); Andreas NICOL, Leverkusen (DE); Torsten FELLER, Leverkusen (DE); Holger SUMMER, Leverkusen (DE); Stefan GOLZ, Leverkusen (DE); Bernd KALTHOF, Leverkusen (DE)

(21) Appl. No.: 18/698,525

(22) PCT Filed: Sep. 30, 2022
(86) PCT No.: PCT/EP2022/077322

§ 371 Date: Apr. 4, 2024

(30) Foreign Application Priority Data
Oct. 6, 2021 (EP) 21201202.5

(51) Int. Cl. C12Q 1/6851; B01L 3/00; B01L 7/00; G01N 35/00

(52) U.S. Cl. CPC C12Q 1/6851; B01L 3/50851; B01L 7/5255; G01N 35/00029; G01N 2035/00158; G01N 2035/00366

(57) ABSTRACT

The invention relates to a system comprising at least one disposable comprising one or more wells in a body, wherein the wells are capable of acting as vessel for one or more chemical, biochemical or molecular biological reactions requiring one or more prescribed reaction temperatures according to a prescribed protocol, said body comprising a flat bottom side building a first heating surface capable of homogeneously conducting heat into the wells and a flat upper side comprising well openings sealed by means of a transparent sealing foil building a second heating surface capable of homogeneously conducting heat in the wells, said body being arranged in a rigid frame support; one or more separate tempering units TUx, each tempering unit TUx comprising at least one first temperature-controlled plates with a planar seat to conform to the flat bottom side of the disposable; transport means comprising a carrier in which the disposable is placed and a moving mechanism for moving the disposable into and/or out of the tempering unit TUx and for contacting one of the heating surfaces of the disposable with the first temperature-controlled plates; a control unit comprising one or more processors configured to activate the tempering units TUx and the transport means for the one or more chemical, biochemical, or molecular biological reactions. The solution of the present invention is particularly adapted for high throughput reactions or assay.

The present invention is directed to an integrated system for chemical, biochemical or molecular biological reactions requiring one or more set point reaction temperatures according to a prescribed protocol, for example real-time polymerase chain reaction or thermal shift assay.

### PRIOR ART

Amplification of DNA by polymerase chain reaction (PCR) and thermal shift assays (TSA) are methods fundamental to molecular biology. In case of PCR they require a sample preparation, amplification of the sample, and product analysis according to a prescribed protocol. Although these steps are usually performed sequentially, amplification and analysis can occur simultaneously. DNA dyes or fluorescent probes can be added to the PCR mixture before amplification and used to analyze PCR products during amplification. Sample analysis occurs concurrently with amplification in the same tube within the same instrument. This combined approach decreases sample handling, saves time, and greatly reduces the risk of product contamination for subsequent reactions, as there is no need to remove the samples from their closed containers for further analysis. The concept of combining amplification with product analysis has become known as “real time” PCR. See, for example, U.S. Pat. No. 6,174,670. Such reactions are typically carried out in a thermal cycler or thermocycler with the capacity to illuminate each sample with a beam of light of at least one specified wavelength and detect the fluorescence emitted by the excited fluorophore. The thermocycler is also able to rapidly heat and chill samples, thereby taking advantage of the physicochemical properties of the nucleic acids and DNA polymerase.

PCR reaction protocols generally comprise a series of temperature changes (thermocycle) that are repeated up to 45-50 times. These thermocycles normally consist of two or three stages: the first, at around 95° C., allows the separation of the nucleic acid's double chain; the second, at a temperature of around 50-60° C., allows the binding of the primers with the DNA template; the third, at between 68-72° C., facilitates the polymerization carried out by the DNA polymerase. This latter may be omitted for small sized fragments. The fluorescence is measured during short temperature phases lasting only a few seconds in each cycle after the second or third stage at corresponding temperature.

The temperatures and the timings used for each cycle depend on a wide variety of parameters, such as the enzyme used to synthesize the DNA, the concentration of divalent ions and deoxyribonucleotides (dNTPs) in the reaction and the melting temperature of the primers.

PCR and qPCR (quantitative PCR) are typically conducted in microplates comprising multiple wells or tubes.

Experiments with customary high-density-microtiter plates—means having a large number of wells—sealed with a thin sealing foil and tempered using heating plates or water baths, do not achieve homogeneous reaction conditions across the whole microplate and are therefore limited, especially for PCR analysis, to endpoint analyzes. Real-time PCR reactions in which a status evaluation of the PCR reaction is taken after each reaction cycle are not satisfactory with these devices. The implementation of a reliable and precise method on samples located in a microplate requires a precise and over the entire microplate uniform application of the process parameters.

In addition to many wells, thermally conductive microplates should therefore be characterized by a homogeneous, high thermal conductivity and stability. This is a particularly challenging requirement for high-density plates with comparatively high number of wells on the one hand and the reproducible quality of well bottom necessary for many applications on the other hand. It is also challenging to provide microplates with well-bottoms, whereby the compaction the material of the bases is even over all the well bases.

US2003/0064508A1 proposes a solution using a microplate made from a thermoplastic polymer formulation comprising a base polymer and a conductive medium, so thermal conductivity is greater than that of the base polymer. The microplate may also include a transparent lid, or cover, preferably made from polycarbonates, polypropylenes, or cyclic olefins or from multi-layer films made from two or more clear materials with desired barrier properties, that may or may not be ultrasonically welded to the plate. A flat piece of conductive material attached to the flat bottom of the plate may be used additionally to impart conductivity and flatness to the part. Alternatively, the flat bottom surface of the plate may be metallized or coated with a flat layer of conductive material. Thermal cycling of a test compound may be accomplished through contact between the microplate holding the reaction medium in a thermocycler comprising a heating block, for example as described in U.S. Pat. No. 5,525,300A, that is rapidly heated and cooled.

There is still a need for a solution allowing the required precise and uniform application of process parameters over an entire microplate, also using a high-density microtiter plate that would address cost, thermal response, and uniformity in particular for the PCR process. The solution shall allow automatization of the reaction processing while conserving form-stability of the microplates as far as possible throughout processing.

### SUMMARY OF THE INVENTION

The problem was solved by a system according to claim 1 and method of use thereof according to claim 15. Exemplary embodiments of the invention can be gathered from the respective dependent claims.

The invention will be elucidated below without distinguishing between the subjects of the invention. On the contrary, the following elucidations are intended to apply analogously to all the subjects of the invention, irrespective of in which context they occur.

The object of the invention is a system for conducting one or more chemical, biochemical or molecular biological reactions in a disposable comprising one or more wells at one or more set point reaction temperatures. The disposable is in particular a microtiter plate or microplate.

The inventive system is basically composed of independent modules which are operatively linked to each other by transport means for transport and placement of a disposable from and into the modules as required for automated implementation of a predefined reaction protocol.

The system of the invention comprises:
at least one disposable comprising the one or more wells in a body, wherein the wells are capable of acting as vessel for the one or more chemical, biochemical or molecular biological reactions requiring one or more prescribed reaction temperatures according to a prescribed protocol, said body comprising a flat bottom side (also called body floor) building a first heating surface capable of homogeneously conducting heat into the wells and a flat upper side comprising well openings sealed by means of a thin transparent sealing foil building a second heating surface capable of homogeneously conducting heat in the wells, said body being arranged in a rigid frame support;
one or more separate tempering units TUx, each tempering unit TUx comprising at least one first temperature-controlled plates with a planar seat to conform to the flat bottom side of the disposable;
transport means comprising a carrier, preferred a carrier frame, in which the disposable is placed, and a moving mechanism for moving the disposable into and/or out of the tempering unit TUx and for contacting one of the heating surfaces of the disposable with the first temperature-controlled plate;
a control unit comprising one or more processors configured to activate the tempering units TUx and the transport means for the implementation of the prescribed protocol for the one or more chemical, biochemical, or molecular biological reactions.

Homogeneous, high thermal conductivity is achieved by using a microplate body comprising wells, wherein the well bases are as thin as possible with a uniform base thickness and shows a high degree of evenness over its floor and all well bases (also called well-bottoms). Stability is achieved by positioning and fixing the body in a frame support; the body and frame support may be welded, glued or riveted, in particular riveted.

In an embodiment, the alignment of all well-bottoms builds a flat well-bottom plane; and the body between the flat well bottom plane and the flat body floor has a bottom-thickness (BT) from 20 to 1000 μm, most preferred from 100 to 400 μm.

In connection with the present invention, the “bottom thickness” in the sense of the application corresponds to the “bottom thickness” as defined in FIG. 3 in relation with the ANSI standard of the Society for Laboratory Automation and Screening with regard to the well bottom elevation, i. e. the average thickness of all well bottoms on a single microplate. The floor thickness is shown as a nominal value [ANSI SLAS 6-2012 (old SBS-6), https://www.slas.org/SLAS.assets.File/public/standards/ASNI SLAS 6-WellBottomElevation.pdf].

In a particularly preferred embodiment, the wells each have a volume of at most 10 μl, in particular 0.3 to 6 μl, in particular 0.5 to 5 μl, in particular 4 μl or 1 μl.

The disposable can be made in any formats and patterns, including least 96, 384, 1536 or 3456 wells, preferred 1536 wells, in the established 96 well format (12×8), 384 well format (24×16), 1536 wells format (48×32) or 3456 well format (48×72) of the ANSI standard of the Society for Laboratory Automation and Screening.

Disposable dimensions can be varied as well as the wall thickness and microwell profile, except for the flat microwell bottom. Preferred microwell section is circular.

In an embodiment, the disposable is a microtiter plate (also called microplate) or a picotiter plate. In connection with the present invention, the format according to the recommendation of the Society for Biomolecular Laboratory Automation and Screening (ANSI/SLABS 1-2004, ANSI/SBLAS 2-2004, ANSI/SBLAS 3-2004 and ANSI/SBLAS 4-2004), in particular the length and width dimensions, in particular the length, width and height dimensions according to the ANSI standard of the Society for Biomolecular Laboratory Automation and Screening (ANSI/SBS SLAS 1-2004, ANSI/SBS SLAS 2-2004, ANSI/SBS SLAS 3-2004 and ANSI/SBS SLAS 4-2004), in particular length 127.76 mm×width 85.48 mm, in particular length 127.76 mm×width 85.48 mm×height 14.35 mm are used.

In an embodiment, the body is made of thermoplastic polymer capable of sealing with the sealing foil, preferred made of polypropylene or COC (Cycloolefin-Copolymer) without added thermally conductive medium. In other words, no thermally conductive medium such as carbon black, thermally conductive ceramic filler or the like is added to the thermoplastic polymer.

In an embodiment, the frame support is made of polycarbonate or polystyrene, preferred polycarbonate.

After reactants are filled into the wells, the disposable is sealed with a transparent thin sealing foil as established in the art. The transparent thin sealing foil may be made from polycarbonate, polypropylene, cyclic olefin or other plastic materials known to those skilled in the relevant art or from multi-layer films made from two or more clear materials with desired barrier properties as well established in the art. Sealing can be achieved by welding of the sealing foil to the body of the disposable.

In each tempering unit TUx, a thermal communication between the disposable and temperature-controlled plate (s) is achieved to thermally process the liquid samples contained therein.

In an embodiment, one or more of the temperature-controlled plate is a Peltier device.

In an embodiment, the Peltier device comprises a thermoconductive plate, on which the disposable rests, and an array of Peltier elements (i.e. the individual physical unit that converts electrical current into heat/cold) contacting the thermoconductive plate.

In an embodiment, the tempering units TUx further comprises a second temperature-controlled plates arranged so that the disposable can be positioned in an internal space between the first and the second temperature-controlled plates and evenly clamped between said plates. In this embodiment, the disposable can be tempered per contact on both heating surfaces.

In an embodiment, the first and the second temperature-controlled plates of a tempering unit TUx are set at the same temperature.

In an embodiment, the temperature of the second temperature-controlled plates is constant during the entire thermocycle at a slightly higher temperature than the temperature of the first temperature-controlled plates. The higher temperature prevents condensation on the sealing foil inside of the well.

In an embodiment, the second temperature-controlled plate for the tempering of the sealing foil is a tempered glass plate. A camera can be used to acquire images through the glass plate, for example for monitoring fluorescence changes, for example during the PCR cycle or other assay formats.

In an embodiment the heating element on the glass plate is an indium tin oxide (ITO) coating. This allows the glass to be heated to more than 100° C., but also reduces the transparency to around 70%.

In a preferred embodiment, a temperature sensor, e. g. a thermal resistor (such as Pt100 sensor) is placed with conductive paste on top of the glass plate and the glass plate is connected to a thermocontroller. This type of heating was shown to achieve more homogeneous temperature profiles.

In an embodiment, the temperatures of the temperature-controlled plates(s), of the Peltier device and/or of the Peltier elements are monitored using temperature sensors, e. g. thermal resistors. For most homogeneous tempering, it is preferred that each Peltier element of a Peltier device is provided with a thermal resistor and each Peltier element is controlled separately.

In an embodiment the system comprises at least one tempering unit TUx per set point reaction temperatures of the prescribed reaction protocol.

In an embodiment the system comprises one tempering unit TUx per set point reaction temperatures of the prescribed reaction protocol and one tempering unit TUx for rapid temperature removal. In a preferred embodiment the tempering unit TUx for rapid temperature removal comprises at least one Peltier device.

In an embodiment, the system comprises an imaging unit I, wherein said imaging unit I comprises an imaging device. Imaging devices may be mechanical, digital, or electronic viewing device, such as still camera, camcorder, motion picture camera, scanner or any other instrument, equipment, or format capable of recording, storing, or transmitting visual images of an object. In an example a CCD camera was used. For example, a scientific Complementary Metal-Oxide-Semiconductor (sCMOS) Camera with a 35 mm F1.6 C-mount objective was used.

In an embodiment the imaging unit I also comprises one or more lighting elements for adequate illumination of the disposable during image acquisition. In an embodiment, a ring light positioned for homogeneous lighting of the disposable over all cavities during image capture is used. Filters may be used.

The person skilled in the art will appreciate that filters depend on use of the device. Among others, excitation and emission filters for image acquisition using photosensitive substances. The imaging unit I may also comprise optical lens(es) and/or mirror(s) for example in case compact device is required.

In an embodiment, several images with different wavelengths can be recorded per thermocycle; for this purpose, a filter changer can be used.

In an embodiment, the imaging unit comprises a support and calibration, means for positioning the imaging device, the lighting elements, the filter changer and/or the filters in relation to the disposable for optimal image acquisition.

In an embodiment the imaging device is positioned to acquire images from the sealed side of the disposable.

In an embodiment the transport means are capable of transporting and positioning the disposable in the imaging unit I for image capture of at least part of the disposable, most preferred of the whole sealed surface of the disposable.

In an embodiment, the imaging unit I comprises a temperature-controlled plates Ix for tempering the disposable from the side opposite the sealing foil.

In an embodiment, the imaging unit I comprises a clamping frame or a transparent clamping plate, so the disposable can be clamped between the temperature-controlled plates Ix and the clamping plate or frame. For this purpose, a clamping mechanism may be used.

In an embodiment, the control unit is configured to control the imaging unit I. Controlling the imaging unit I comprises activating, deactivating or positioning the imaging device, the lighting device, the filters and/or the clamping mechanism as needed for optimal image acquisition.

In an embodiment, the transport means comprises a moving mechanism for moving the disposable into and/or out of the internal space of the tempering unit TUx and/or of the imaging unit Ix, said internal space being defined as the contact space of the first temperature-controlled plates, the internal space between the first and the second temperature-controlled plates in a tempering unit TUx or the contact space between the temperature-controlled plates Ix and the clamping plate or frame in the imaging unit I (FIG. 4).

In an embodiment the transport means comprises at least one horizontal drive and the disposable is positioned on the moving carrier for transport and positioning in the tempering unit TUx and imaging unit I as required by the reaction protocol.

In some embodiments, the carrier can be movable between the internal microplate position and an external microplate position outside an instrument casing for loading and/or unloading the microplate to/from the carrier. Specifically, in some embodiments, the carrier is movably mounted to a base for performing a repetitive, bidirectional movement between the internal and external disposable positions. In some embodiments, the moving mechanism is configured as carrier driving mechanism for driving the carrier in either of the two directions, means for driving the carrier into the internal and external microplate positions, respectively.

In an embodiment, the carrier is slidably mounted to the horizontal drive(s) enabling a repetitive, bidirectional movement between the processing positions inside the system for thermally processing the reaction products and a loading position outside the system for loading or unloading the disposable on/from the carrier. Since such sliding mechanism is well-known to those of skill in the art, it need not be further elucidated herein. In some embodiments, the system comprises an automated carrier driving mechanism such as a motor-based belt- or wheel-drive for automatically moving the carrier between the processing and loading positions. Since such driving mechanism is well-known to those of skill in the art, it need not be further elucidated herein. In an embodiment, horizontal and vertical motors, e. g. servo or stepper motors, are used to transport the microplate assembly (means microplate and carrier) to contact with the top surfaces of the temperature-controlled plates.

In an embodiment the disposable is positioned in the carrier on a metal heating fixture shaped to closely conform to the disposable, in particular to its frame support, and to the temperature-controlled plates(s).

In an embodiment, the system comprises one or more clamping mechanisms for clamping the disposable in the internal space of the tempering units TUx and/or of the imaging unit I.

In a tempering unit TUx the clamping mechanism is capable of moving one of the temperature-controlled plates in relation to the other temperature-controlled plates to allow clamping the disposable between the first and the second temperature-controlled plates after the disposable is positioned between these by the transport system.

Accordingly, a full contact on both sides of the disposable can advantageously be obtained by clamping.

In the Imaging unit I, the clamping mechanism can move the temperature-controlled plates in relation to the clamping plate/frame for positioning of the disposable in the imaging unit I.

It is preferred that at least one of the temperature-controlled plates in a tempering unit TUx or one of the temperature-controlled plates or the clamping plate/frame in the imaging unit I is spring-mounted for smooth clamping of the disposable. In an embodiment, a force measurement may be implemented using the spring constant and the stroke of the spring-mounted element.

In an embodiment, the same clamping mechanism is used for clamping the disposable in the tempering units TUx and in the imaging unit I. In this embodiment, spacing/alignment blocks can be used to achieve aligned positioning of the disposable in the units, in particular by way of aligning the planar seats (contact surfaces) of the temperature-controlled plates(s) of the tempering units TUx and/or imaging unit I. In an embodiment, temperature-controlled plates may be mounted on a spacing/alignment block for better positioning.

In case a tempering unit comprising Peltier device(s) is used, it is preferred that basic height for the aligned positioning of the disposable in the system of the invention is given by the position of the Peltier device and that spacing/alignment blocks provide consistent height control across the further tempering units TUx and/or imaging unit I.

In an embodiment clamping of the disposable is achieved by a vertical motor moving the lower temperature-controlled plates upwards and pressing the disposable against a spring-mounted upper temperature-controlled plates. A force measurement may be implemented using the spring constant and the stroke of the upper temperature-controlled plates.

The person skilled in the art will appreciate that further embodiments for clamping the disposable in the internal space of a unit may be used. For example, means for clamping can cause the disposable to be pressed down onto the lower temperature-controlled plates.

In an embodiment, the control unit is configured to activate the first temperature-controlled plates, the second temperature-controlled plates, the transport means and/or the clamping mechanism according to the predefined reaction protocol.

In an embodiment, the system comprises more than one tempering units TUx at different temperatures and temperature changes are achieved by transporting and positioning the disposable in the tempering unit TUx at adequate temperature by way of the transport means and the clamping mechanism if used. In an embodiment a tempering units TUx comprising at least one Peltier element is used, in particular for heat removal from the disposable. Compared to a static embodiment (one temperature-controlled heating/cooling plates), this embodiment has the advantage of not having to wait for the temperature-controlled plates to transition to next set point temperature, which can speed up test time significantly.

In an embodiment, the disposable is made of white thermoplastic polymer. Although typical qPCR disposables are often white, fluorescent assays are also conducted and measured in black disposables through the sealing foil. The material properties of the white disposables were shown to be advantageous also for imaging of fluorescent assays (FIG. 13). Monochrome pictures taken with the same camera settings (0.5 s exposure time) and displayed with a color palette picture, show that a white plate shows a higher fluorescent signal even in the empty wells, but much higher in the wells with the fluorophore. In the picture of a black plate under the same conditions, there is no signal/no difference between wells or walls. If the optical fibers are positioned directly above the plate and illuminated perpendicularly into the wells, then a weak fluorescent signal is detectable. Since the wells are relatively deep, the diagonal incident light does not appear to hit the fluorophore (small volume).

The white material has a high autofluorescence and reflection in contrast to the black one. This leads to stronger background noise, but at the end the signal to noise ratio was found to be more important.

In an embodiment, the system of the invention may also comprise means for illumination of oligonucleotides adapted to induce cleavage of a photolabile chemical bonding, preferably in a separate module adapted for control of reaction conditions.

In an embodiment, the system of the invention may comprise a module for providing all reagents necessary for the reaction at in the right concentration into the disposable. As unit for providing all reagents within the present invention, a pipetting robot or a pipetting unit can be used.

In some embodiments, the system of the invention is an instrument for incubating, thermally treating or otherwise processing liquid samples such as an automated thermocycler enabling liquid reaction mixtures to be put through a series of temperature excursions, e.g., for performing the PCR or any other reaction of the nucleic acid amplification type.

In an embodiment, the chemical or biochemical reactions are selected from the group comprising a primer extension reaction such as Nucleic acid sequence-based amplification, commonly referred to as NASBA, rolling circle amplification, analytical polymerase chain reaction amplification, in particular polymerase chain reaction amplification in real time, i. e. a qPCR, real time PCR or LAMP (loop-mediated isothermal amplification).

In some embodiments, the instrument is being used for chemically processing liquid samples, e.g., by performing tests or assays related to immunochemical or clinical-chemical analysis items.

To perform all these steps, the system typically requires a user programmable computer system which is configured for controlling the inventive system through a control unit.

In an embodiment the user enters the reaction protocol into the inventive system or selects adequate reaction protocol from a data repository (for example a file or a database) comprising a collection of reaction protocols by way of a user interface.

A further object of the invention is a method for use of the system of the invention described above, comprising the following steps:
loading a reaction mixture in the wells of a disposable and sealing the upper side of the disposable with a sealing foil;
introducing the sealed disposable in the system, transporting and positioning it in one of the tempering units TUx in accordance with the prescribed protocol.

In a further embodiment, the method further comprises transporting to and positioning the disposable in imaging unit I and capturing one or more images of at least one well of the disposable.

It is preferred that one tempering unit TUx is used per prescribed reaction temperature of the reaction protocol and at least one of the temperature-controlled plates in the tempering unit TUx is set at one of the prescribed reaction temperatures.

It is also preferred that the temperature of the second temperature-controlled plates in a tempering unit TUx is set at a slightly higher temperature than the highest prescribed reaction temperature of the reaction protocol.

The solution of the invention is particularly useful for conduction polymerase chain reaction, real-time polymerase chain reaction or thermal shift assay.

In an example the present invention is used for qPCR. This example is explained in somewhat greater detail below, but without any intention of restricting the invention to this embodiment.

The system of the invention can be configured to conduct one or more PCR thermocycle. In an example, the system comprises three tempering units TUx and an imaging unit Ix (FIG. 4).

The present invention also provides methods for performing a real time PCR comprising the steps of
(i) performing an oligonucleotide synthesis of a first primer and a second primer in the system of the invention,
(ii) adding into a disposable positioned in said system
   a. the nucleic acids sample to be analyzed,
   b. said first and second primer synthesized in step (i) and
   c. all other reagents necessary for a real time PCR of said target nucleic acid and
(iii) amplifying and simultaneously monitoring amplification of the target DNA in said system.

In an embodiment primer extension reaction such as NASBA or rolling circle amplification or in particular an analytical polymerase chain reaction amplification in real time, i. e. a qPCR or real time PCR may be performed in the system of the invention.

The solution of the invention is particularly useful for automatically conducting chemical, biochemical or molecular biological reactions in particular high throughput reactions and assays.

The use of terms “a” and “an” and “the” and similar referents in the context of describing the invention (especially in the context of the following claims) are to be construed to cover both the singular and the plural, unless otherwise indicated herein or clearly contradicted by context. The terms “comprising”, “including”, “having”, and “containing” are to be construed as open-ended terms (i.e. meaning “including but not limited to”) unless otherwise noted.

The use of any and all examples, or exemplary language (e.g., “such as”) provided herein, is intended merely to better illuminate the invention and does not pose a limitation on the scope of the invention unless otherwise claimed. No language in the specifications should be constructed as indicating any non-claimed element as essential to the practice of the invention.

Preferred embodiments of this invention are described, including the best mode known to the inventors for carrying out the invention.

Variations of those preferred embodiments can become apparent to those of ordinary skilled in the art to employ such variations as appropriate, and the inventors intend for the inventions to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### BRIEF DESCRIPTION OF THE DRAWINGS

FIG. 1 shows a 3-dimensional view of a disposable according to the invention wherein the body 150 riveted to the frame support 300.

FIG. 2 is a cross sectional view of the disposable according to FIG. 1

FIG. 3 shows an enlargement of FIG. 2 wherein characteristic measurements of the disposable are defined.

### LIST OF REFERENCE NUMERALS

1 disposable
150 body of the disposable
151 well
152 well wall
153 well bottom
154 body floor
156 planar top
161 cams
200 flat well-bottom plane
300 frame support
301 rivet
WBE—WELL BOTTOM ELEVATION
WD—WELL DEPTH
ECTP—EXTERNAL CLEARANCE TO PLATE BOTTOM
WBW—WELL BOTTOM WIDTH
BT—BOTTOM THICKNESS
IWBEV—Intra-Well Bottom Elevation Variation

FIG. 4 is a schematic representation of the system of the invention comprising three tempering units TU1 to TU3 and an imaging unit I. The transport means are represented by the horizontal arrows; the clamping mechanism is not represented.

FIG. 5 shows an oblique montage diagram of a system according to the invention without disposable-

### REFERENCE NUMERALS

I imaging unit
20 imaging device
21 light (optical fibers, lamp not shown or light ring)
22a excitation filter, 22b emission filter
23 clamping frame or clamping plate
24 Spacing/alignment block for temperature-controlled plates
25 means for clamping (vertical motor 25a+vertical drive 25b, tooth belt 25c)
26 temperature-controlled plates of the imaging unit I+positioning means for disposable 1
TU1-TU4 tempering units
30, 31 temperature-controlled plates, wherein 30a, 31a are fixe temperature plates of tempering units TU1 to TU3 and 30b, 31b are Peltier devices of tempering units TU4,
32 Spacing/alignment block for temperature-controlled plates
33 means for clamping
40 load position
41 carrier/carrier frame
42 motor
43 horizontal drive
50 support frame

FIG. 6 shows the heating curve of the microplate with heating from 60° C. to 95° C. over time obtained for the embodiment of disposable 1 described above and measured using a thermography camera Type FLIR 645 sc (Long-Waved Infrared LWIR).

FIG. 7 shows a plot of a measurement obtained using a sCMOS Camera with a 35 mm F1.6 C-mount objective which shows the homogeneous amplification over the entire plate.

FIG. 8 shows a side view diagram of a particular embodiment of a Peltier device 50.
50 Peltier device
51 thermoconductive plate, on which the disposable rests
52 Peltier elements in array
53 heat sink
55 active microplate area
56 sensor (measuring only)
57 sensor as actual value

FIG. 9 and FIG. 10 show the schema a Peltier device 50 comprising an array of six Peltier elements 52 equipped with Pt100 sensors, with different control settings (9A, 10A); FIGS. 9B and 10B show the temperature deviation of the individual Pt100 sensors from the setpoint of the respective Peltier 50 device depending on control setting.

FIG. 11 shows an oblique montage diagram of an imaging unit I as used for TSA experiment.

FIG. 12 shows a plot of a fluorescence TSA measurement of a homogeneously filled 1536 plate 1 acquired with the sCMOS camera, wherein image 12A shows the calculated Tm values in the form of a heat map, while FIG. 12B shows the measured curves used to calculate the Tm values (position of the inflection point).

FIG. 13 shows a fluorescence measurement acquired with the sCMOS camera using a black microplate (FIG. 13A) compared to a measurement obtained using a white microplate (FIG. 13B).

### DETAILED DESCRIPTION OF THE ILLUSTRATED EMBODIMENT

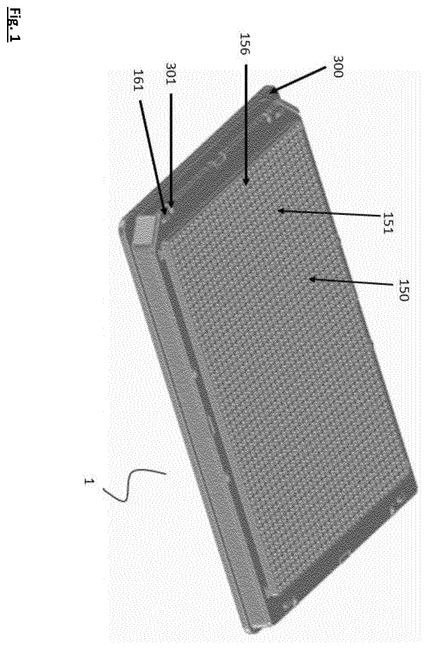

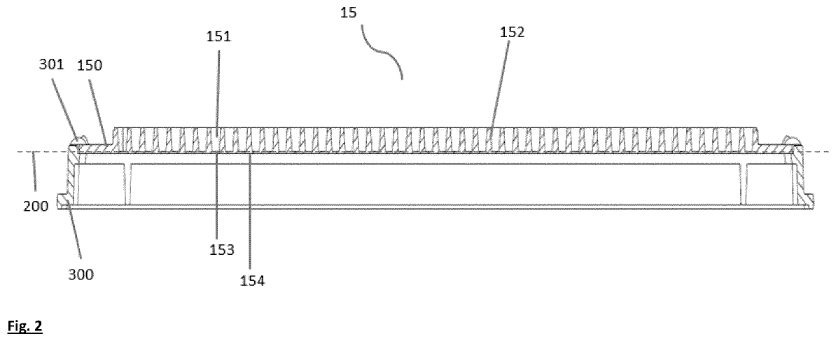

FIG. 1 illustrate a disposable 1 comprising a body 150 and a plurality of microwells 151 recessed in the body 150. The body 150 is riveted in frame support 300 by way of rivets 301. The frame support 300 provides structural support to the body 150 during transport and when vertical load is applied by the clamping mechanism. The body 150 shows cams 161 to improve the stack ability of the plates.

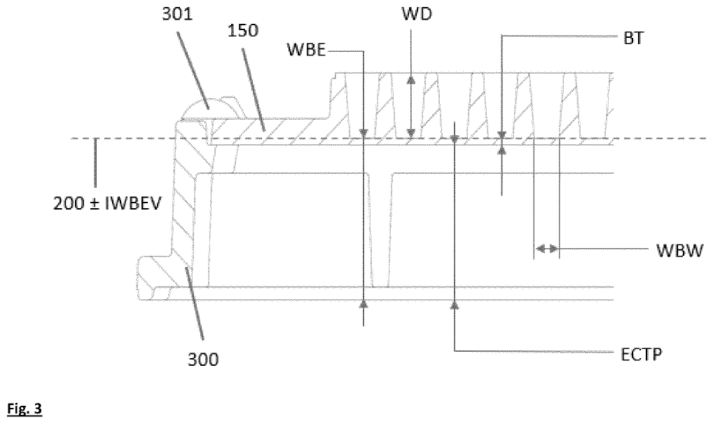

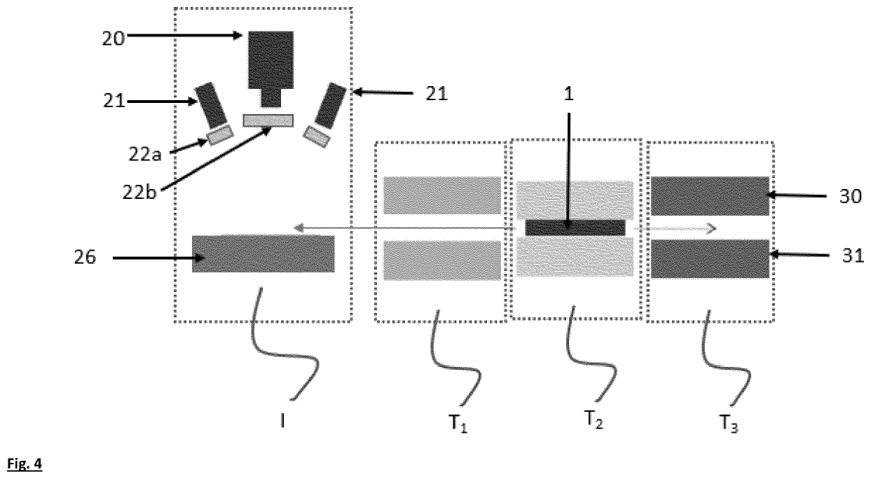

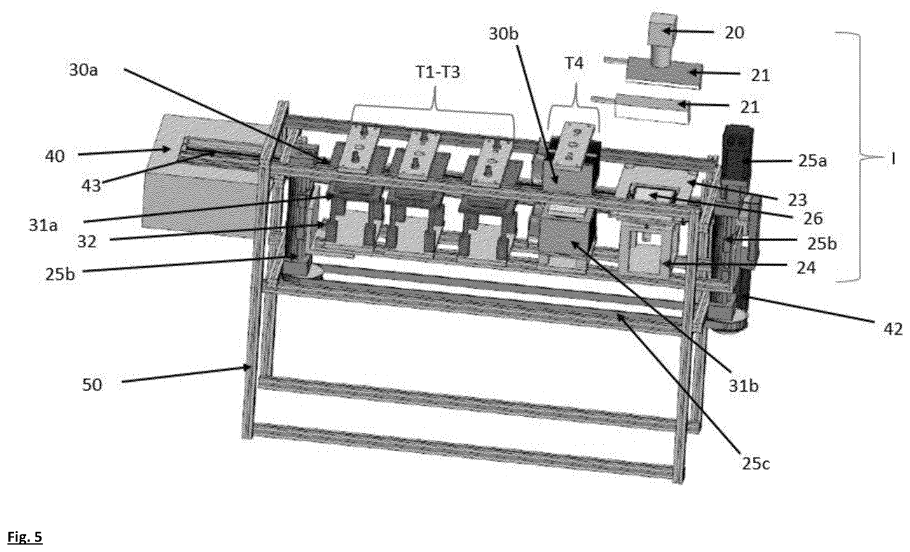

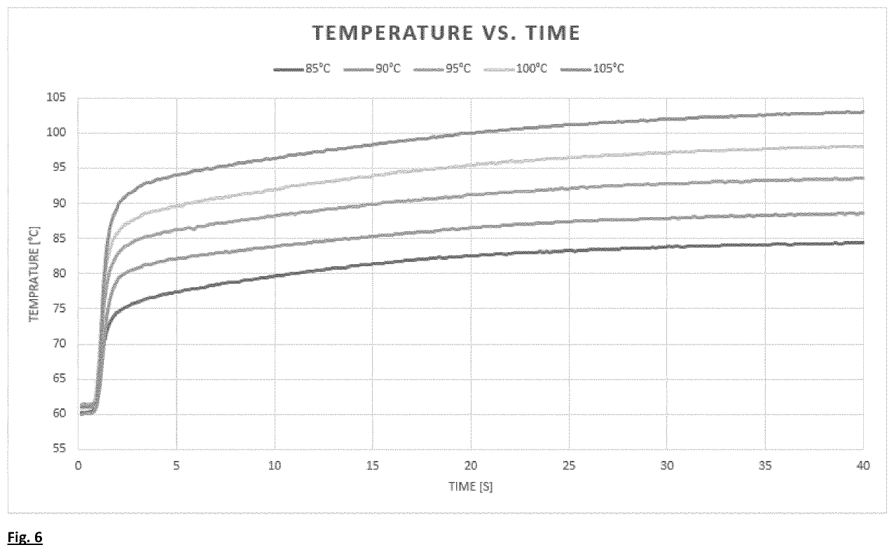

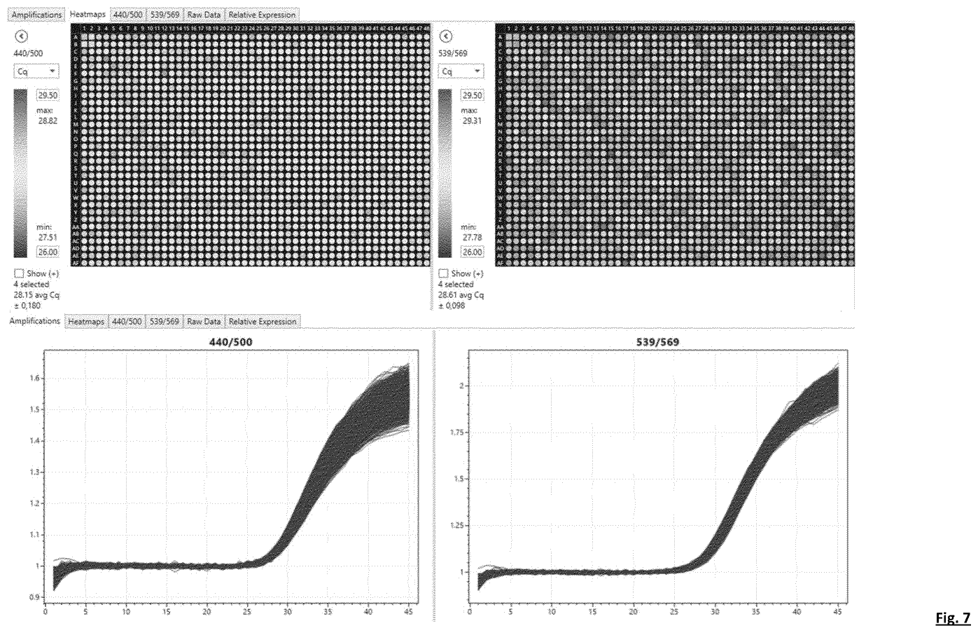

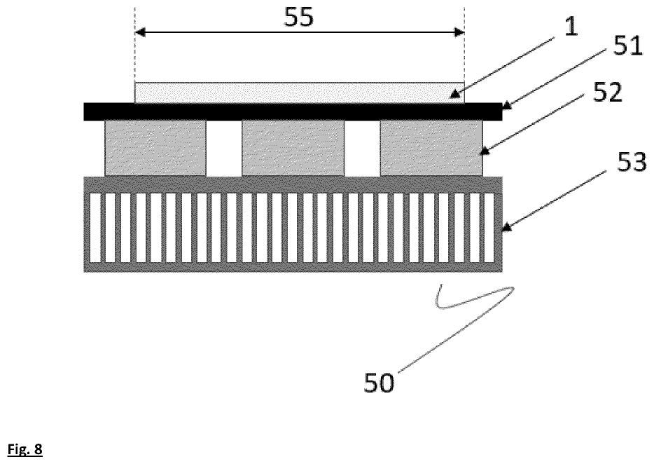

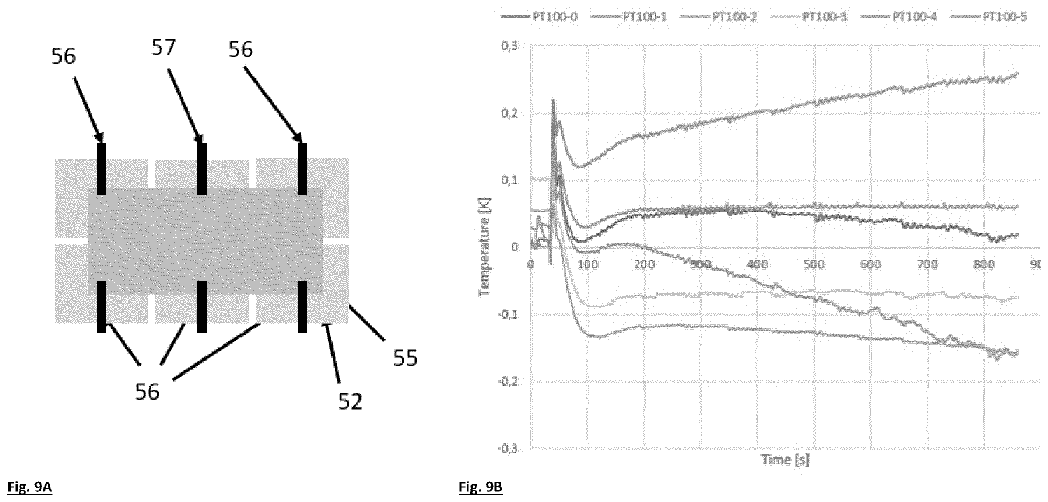

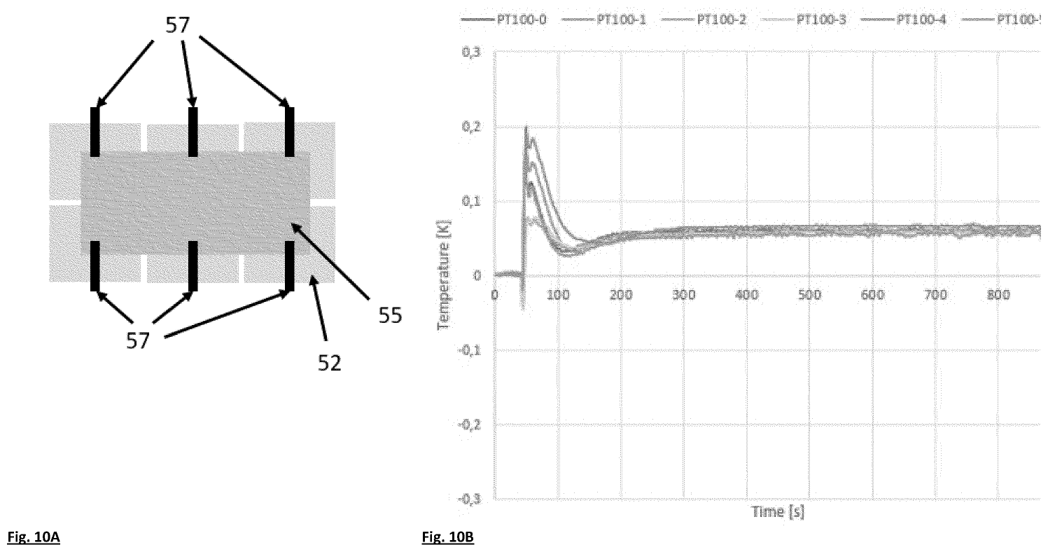

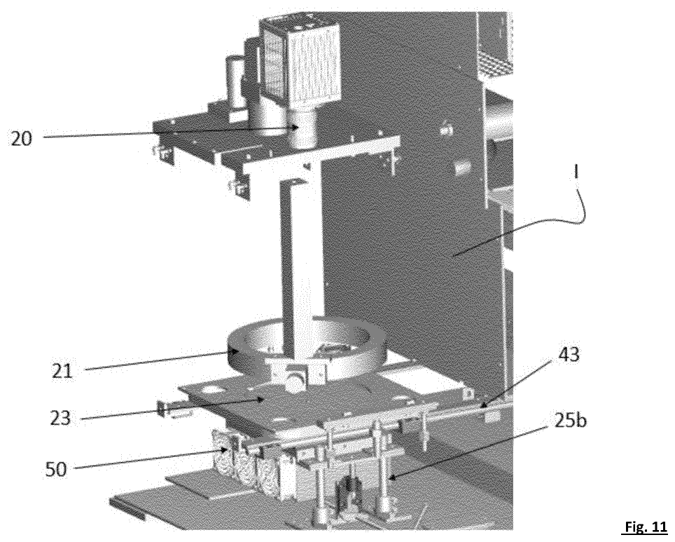

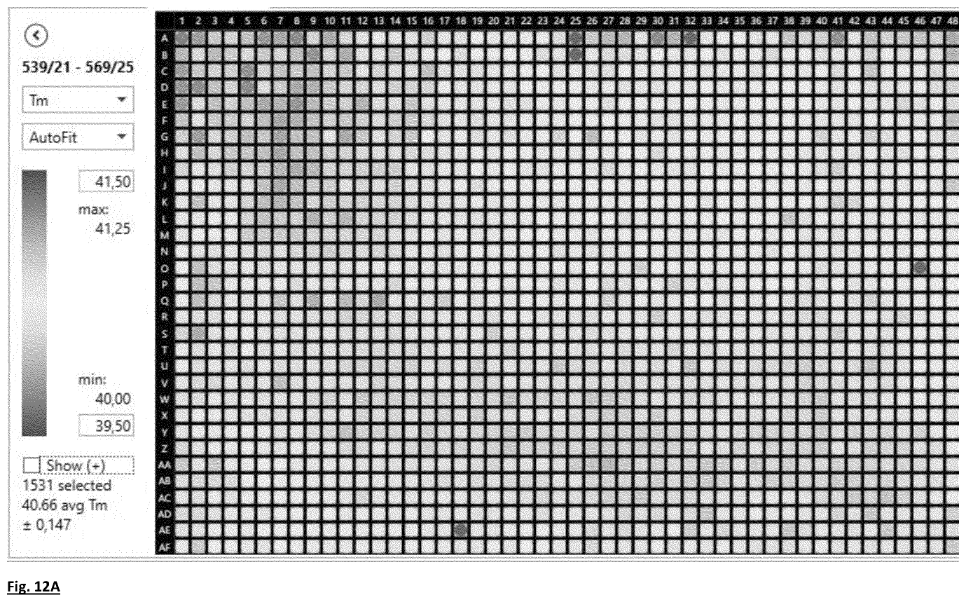

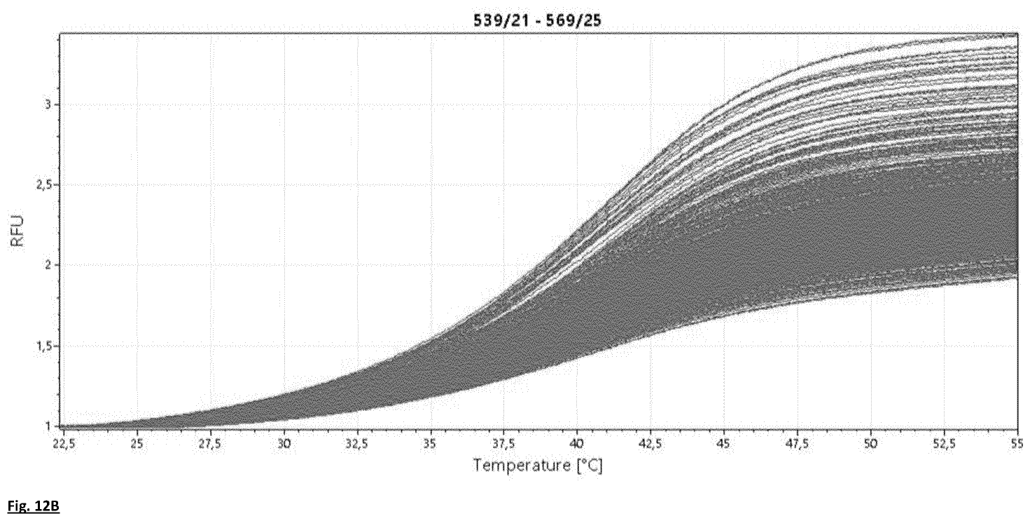

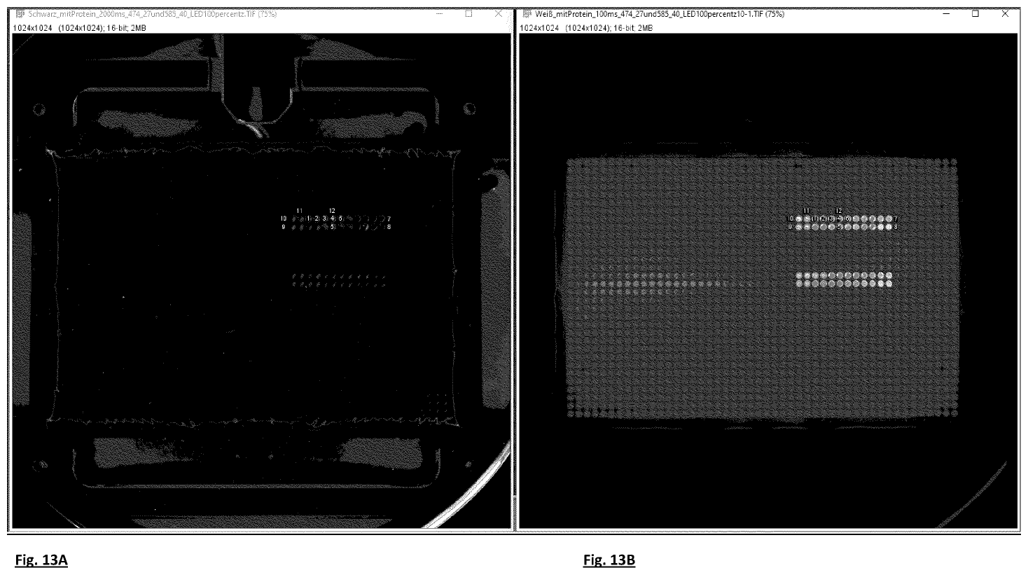

FIG. 2 illustrates the body of the disposable 150 in a frame support 300 in cross sectional view. The microwell side wall 152 has a profile that is substantially cylindrical or conical with a planar microwell bottom 153. The alignment of the flat microwell bottoms 153 build a well-bottom plane 200. A bottom-thickness BT between well-bottom plane 200 and body floor 154 of 350 μm was used. The body 150 shows male rivets 301 for fixation to the frame support 300. FIG. 3 shows an enlargement of FIG. 2 wherein characteristic measurements of the disposable are defined. When load is applied vertically down the microplate 1, body floor 154 is pressed towards the top surface of temperature-controlled plates to make intimate contact. In case the body floor 154 is slightly concave/convex, it flattens out nicely, when vertical load is applied.

FIG. 4 is a schematic representation of the system of the invention comprising three tempering units TU1 to TU3 and an imaging unit I. The transport means are represented by the horizontal arrows; the clamping mechanism is not represented. The simplified imaging unit I comprises imaging device 20 and a light ring 21 for homogeneous illumination of the disposable 1 for image acquisition. The temperature-controlled plates 26 of the imaging unit I is at desired temperature T when disposable 1 is positioned in the imaging unit I. Tempering units TU1 to TU3 comprise a first/lower and a second/upper temperature-controlled plates 31, 30. During operation of the system of the invention, the disposable 1 is positioned in one or the other tempering unit TUx and clamped between the temperature-controlled plates 30, 31 for a period as prescribed by the experiment protocol, then transported to the next tempering unit for further reaction or to the imaging unit I for image acquisition.

In an embodiment, the disposable 1 is configured to engage with the temperature-controlled plates dynamically where it attaches and detaches throughout the PCR test process. Multiple temperature controlled heating/cooling plates 31a, 31b, 31c set to different temperatures are provided, in this example, the first plates 31a has a uniform temperature T1 (from 90° C. to 98° C., for example) to studying varying denaturation temperature effect; the second plates 31b is set at uniform temperature T2 (50° C., for example) for annealing process; the third plates 31c is set at uniform temperature T2 (70° C., for example) for extension process. It is preferred that the number of temperature-controlled heating/cooling plates matches the number of temperature set points of a thermal cycle.

FIG. 5 shows an oblique montage diagram of the system of the invention without disposable comprising three tempering units TU1 to TU3 with fixe temperature, a tempering unit TU4 comprising Peltier plates 30b, in particular for temperature removal (cooling) from the disposable 1 as needed, and an imaging unit I.

Disposable 1 is transported between a load position 40 for the introduction of disposable 1 in the device and the units using a horizontal transport means, by placing said disposable 1 on a carrier 41 (not shown) movable on horizontal drive 43 using a horizontal stepper motor 42. Vertical motor 25a is mechanically engaged with vertical drives 25b by way of tooth belt 25c. During experiment, driven by an automated software program in a control unit (not shown), disposable 1 engaged in carrier 41 is transported by the horizontal motor 42 (a stepper motor was used) along the horizontal drive 43 to specific temperature zone, then it is pressed and held against the top surface of one of the temperature controlled plates by clamping means 25 to allow the chemical reaction to take place. It is then moved to the next temperature zone for next temperature set point in the thermal cycle.

Means for clamping can include any vertical load application mechanism used to cause the disposable 1 to be pressed up onto the clamping frame/plate or the upper temperature-controlled plates 30, alternatively pressed down onto the lower temperature controlled plates.

In the embodiment of FIG. 5, clamping of disposable 1 is achieved by vertical clamping means 25, 33, moving the lower temperature-controlled plates 26, 31a and spacing/alignment block 24, 32 and lower temperature-controlled plates 31b upwards and pressing the disposable 1 against spring-mounted clamping frame 23 (in imaging unit I) or upper temperature-controlled plates 30a, 30b respectively. A force measurement may be implemented using the spring constant and the stroke of the upper temperature-controlled plates (not shown). In this embodiment basic height for the position of disposable 1 in the device is given by the position of the Peltier elements 30b, 31b in tempering unit TU4; spacing/alignment blocks 24, 32 align microplate 1 to the temperature-controlled plates 26, 31a and provide consistent height control across the microplate 1 when vertical load is applied onto the microplate 1. Spacing/alignment blocks 24, 32 can be made using a structurally stable material, such as, but not limited to, stainless steel.

The imaging unit I comprises imaging device 20, a light ring 21, both directed downwards the disposable 1 and filters 22 mounted on a frame (not shown) for adequate image acquisition. When transported into the imaging unit I, disposable 1 is clamped between the temperature-controlled plates 26 and spring-mounted clamping frame 23 as described above.

Clamping frame 23 may be a stiff plate with large rectangular cutout in the center to allow optical access for all microwells 151 in the microplate 1. It is preferably fabricated from stiff material such as, but not limited to, stainless steel. Clamping transparent plate is preferably made of stiff material, such as, but not limited to, quartz glass, sapphire, and the like. It is surface should be sufficiently planar.

In embodiments where space constraint is a concern, the optical imaging module may comprise one or more surface mirrors for space optimization.

FIG. 6 shows the heating curve over time of microplate 1 heated from 60° C. to 95° C. in a test system comprising an imaging unit I with a thermography camera Type FLIR 645 sc (LWIR), a temperature-controlled plates and a clamping glass plate. With a temperature jump of nominally 35K, the increase amounts to 20K in approx. 2 s. This thermal experiment shows that required rapid heat transfer from the temperature-controlled plates into microplate 1 is achieved.

The system of FIG. 5 was tested for high throughput qPCR experiments.

In an example, the following steps were carried out in a white disposable/microplate 1 according to the invention with 1536 wells, WBE=7.4 mm, WD=3 mm, WBW=1.2 mm, BT=0.35 mm, ECTP=7.1 mm comprising a body made of polypropylene in a frame support of polycarbonate produced by company Greiner Bio-One GmbH.

A mixture of the following solutions was used for each well 151 of microplate 1 and pipetted into the wells:
Solution per well UltraPlex™ 1-Step ToughMix® (4×)—Quantabio 0.25 μl.
qPCR Human Reference cDNA, random-primed (TaKaRa Bio 639654) 0.05 ng
RPL32 primers and probe mixture (stock concentration of equimolar mixture 1.33 μM) 0.25 μl as well as RPL30 primers and probe mixture (stock concentration of equimolar mixture 1.33 μM) 0.25 μl.
PCR suitable water add 1 μl.

The microplate 1 was then sealed with an optically clear, permanently adhesive film (Applied Biosystems, 4311971). The microplate 1 was centrifuged and introduced into the system of FIG. 5 for real-time PCR measurements by way of rapid heating or cooling of microplate 1 according to the implementation protocol described below.

A system comprising tempering units TU1, TU2 at temperatures T1 (95° C.) and T2 (72° C.) respectively and a tempering unit TU3 comprising a Pelter device at T3 (=60° C.) for temperature removal as needed were used as following:

After an initial time of 2 minutes at 95° C. in tempering unit TU1, a cycle of 3 temperature steps with first 10 seconds 95° C. (tempering unit TU1), second 30 seconds at 60° C. (tempering unit TU3) and third 5 seconds 72° C. (tempering unit TU2) was conducted. This cycle was repeated 45 times. In each cycle, after the third step (72° C.), the disposable was transported into the imaging unit I and positioned on a temperature-controlled plate at 72° C., irradiated/excited with light with a wavelength of 539 nm and the light emitted again at 569 nm was recorded using a sCMOS Camera (Hamamatsu, ORCA-Flash4.0 V3, C13440-20CU) with a 35 mm F1.6 C-mount objective.

### Overview of the Primers/Sample Used for the PCR Reaction:

### Description of Gene Sequences

539/569:
RPL32_forward
5′-GCACCAGTCAGACCGATATGT-3′
RPL32_reverse
5′-ACCCTGTTGTCAATGCCTCT-3′
RPL32_Probe
5′-AATTAAGCGTAACTGGCGGAAACCC-3′
(5′ labeled with the Fluorophor HEX and 3′
labeled with the quencher BHQ1)
and
440/500:
RPL30_forward
5′- GTCCCGCTCCTAAGGCAG -3′
RPL30_reverse
5′- GTTGATCGACTCCAGCGACT -3′
RPL30_Probe
5′- AGATGGTGGCCGCAAAGAAGACGAA -3′
(5′ labeled with the Fluorophor Cyan500
and 3′ labeled with the quencher BHQ1)

FIG. 7 shows a plot of a fluorescence measurement acquired with the sCMOS camera, which shows the homogeneous amplification over the entire plate.

In an embodiment, a Peltier device comprising a thermoconductive plate, on which the disposable rests, and an array of Peltier elements (i. e. the individual physical unit that converts electrical current into heat/cold) contacting the thermoconductive plate is used.

Most preferred is an array of six Peltier elements, considering MTPs have an aspect ratio of 3:2.

In an embodiment, the Peltier device also comprises a heat sink that dissipates the entire electrical power converted and carries the Peltier elements. Also, a heatsink/fan combination can be used.

The thermoconductive plate(s) is preferably made of metal, such as aluminum.

It is preferred that each Peltier element of the array is monitored using temperature sensors, for example thermal resistors, such as Pt100 sensors.

FIG. 8 shows a diagram of a particular embodiment of a Peltier device 50, comprising a thermoconductive plate 51, on which the disposable 1 rests, and an array of Peltier elements 52, and a heat sink 53.

The recorded temperature values recorded by the temperature sensors (56/57) are used by the control unit configured for regulating the constant temperature of the heating blocks at the temperature(s) as required by the experiment protocol. For most homogeneous tempering, it is preferred that each Peltier element 52 of a Peltier device 50 is provided with a thermal resistor and each Peltier element 52 is controlled separately. FIG. 9 and FIG. 10 show a comparison of the temperature deviation of a Peltier device 20 comprising six Peltier elements 52 equipped with Pt100 sensors depending on control setting. In FIG. 9, control is achieved with one Pt100 sensor as actual value 57 for the Peltier device, while other Pt100 sensors 56 are only measuring temperature of their respective Peltier elements. In comparison, FIG. 10 shows an embodiment wherein control is achieved with one Pt100 sensor as actual value 57 for each Peltier element 52 of the Peltier device 50, clearly showing improved homogeneous temperature over the microplate.

In an embodiment, the Peltier device of FIG. 8 was found to be most preferred for experiments wherein the entire disposable 1 is to be heated up with a temperature ramp, which must happen simultaneously at all positions of the microplate 1. For example, in a TSA experiment, the entire plate 1 is heated up with a temperature ramp of e. g. 0.04K/s.

It is most preferred that the area spanned by the 6 Peltier elements is significantly larger than the active area of the microplate 55. This embodiment was found to ensure better homogeneity and minimized edge effects.

A system comprising a Peltier device 50 according to FIG. 8 for tempering the microplate 1 from below was tested for high throughput TSA experiments.

For this purpose, an imaging unit I as shown in FIG. 12 was used, said unit I comprising:
the Peltier device 50 described above for tempering the floor of the microplate 1;
a tempering transparent glass plate 23 for contacting the microplate 1 on the sealed side;
clamping means 23 comprising a vertical drive and a vertical motor for clamping the microplate 1 when positioned in the internal space between Peltier device 50 and glass plate 23 by way of transporting along horizontal drive 43; and
an imaging device 20 for imaging the microplate 1 illuminated with ring light 23 from the sealed side.

In an example, the following steps were carried out in a white disposable/microplate 1 according to the invention with 1536 wells, WBE=7.4 mm, WD=3 mm, WBW=1.2 mm, BT=0.35 mm, ECTP=7.1 mm comprising a body made of polypropylene in a frame support of polycarbonate produced by company Greiner Bio-One GmbH.

A mixture of the following solutions was used for each well 151 of microplate 1 and pipetted into the wells:
200 nM final concentration of a 5′-HEX fluophor and 3′ Quencher (BHQ1) labeled RNA Oligo [5′ HEX-CUUGACUGGCGUCAUUCAGGAGCUGGAUGGCGUGGGACAU-BHQ1-3′], 0.05 μl buffer (10 mM Tris-HCl, 1 mM EDTA, 200 mM KCL; pH 7.5)
RNAse/DNAse free water add 1 μl.

The microplate 1 was then sealed with an optically clear, permanently adhesive film (Applied Biosystems, 4311971). The microplate 1 was centrifuged and introduced into the system for TSA measurements by way of rapid heating or cooling of microplate 1 according to the implementation protocol described below.

The disposable was placed into the imaging unit I of FIG. 11 and clamped on the temperature-controlled Peltier device 50. The Peltier device was heated from 28° C. to 55° C. with a temperature increment of 0.04° C./s. The plate was irradiated/excited with light with a wavelength of 539 nm and the light emitted again at 569 nm was recorded using a sCMOS Camera with a 35 mm F1.6 C-mount objective with 25 images per ° C.

FIG. 12 shows a plot of a fluorescence measurement acquired with the sCMOS camera, which shows a plot from a TSA measurement for the Tm determination (position of the inflection point, graph 12B) of a homogeneously filled 1536 plate. The heatmap (12A) over the entire plate shows a temperature distribution (Max-Min) of just 2K across the entire plate.

FIG. 13 shows a fluorescence measurement acquired with the sCMOS camera using a black microplate (FIG. 13A) compared to a measurement obtained using a white microplate (FIG. 13B). Surprisingly, white microplate was found to be better suited for fluorescence read-out.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the scope of the appended claims.

1. A system comprising:
at least one disposable plate comprising one or more wells in a body, wherein the wells are capable of acting as vessel for one or more chemical, biochemical or molecular biological reactions requiring one or more prescribed reaction temperatures according to a prescribed protocol, said body comprising a flat bottom side building a first heating surface capable of homogeneously conducting heat into the wells and a flat upper side comprising well openings sealed by means of a transparent sealing foil building a second heating surface capable of homogeneously conducting heat in the wells, said body being arranged in a rigid frame support;
one or more separate tempering units TUx, each tempering unit TUx comprising at least one first temperature-controlled plates with a planar seat to conform to the flat bottom side of the disposable plate;
transport means comprising a carrier in which the disposable plate is placed and a moving mechanism for moving the disposable plate into and/or out of the tempering unit TUx and for contacting one of the heating surfaces of the disposable plate with the first temperature-controlled plates; and
a control unit comprising one or more processors configured to activate the tempering units TUx and the transport means for implementation of the prescribed protocol for the one or more chemical, biochemical, or molecular biological reactions.

2. The system according to claim 1, wherein the multiple wells have flat well-bottoms, the alignment of all well-bottoms builds a flat well-bottom plane and the body between the flat well bottom plane and the flat body floor has a bottom-thickness BT from 20 to 1000 μm.

3. The system according to claim 1, wherein the body is made of polypropylene or cycloolefin-copolymer without added thermally conductive medium.

4. The system according to claim 1, wherein at least one of the temperature-controlled plates is a Peltier device.

5. The system according to claim 4, wherein the Peltier device comprises a thermoconductive plate, on which the disposable plate rests, and an array of Peltier elements contacting the thermoconductive plate.

6. The system according to claim 1, wherein the tempering units TUx further comprises a second temperature-controlled plates positioned so that the disposable plate can be positioned and evenly clamped between the first and the second temperature-controlled plates.

7. The system according to claim 6, wherein the second temperature-controlled plates is a tempered glass plate.

8. The system according to claim 1, comprising at least one tempering unit TUx per prescribed reaction temperature.

9. The system according to claim 1, further comprising an imaging unit I, wherein said imaging unit I comprises, an imaging device capable of capturing an image of at least one well of the disposable plate from the upper side and a clamping mechanism for precise positioning of the disposable plate when placed in the imaging unit I.

10. The system according to claim 9, wherein the imaging unit I further comprises a temperature-controlled plates capable of tempering the disposable plate from the bottom side during imaging.

11. The system according to claim 9, wherein the imaging unit I further comprises a clamping frame or plate allowing image capture of the one or more wells of the disposable plate, positioned so that the disposable plate can be positioned and evenly clamped between the temperature-controlled plates and the clamping frame or plate in the imaging unit I.

12. The system according to claim 9, wherein said imaging unit I further comprises a ring light positioned for homogeneous lighting of the upper side of the disposable plate during image capture.

13. The system according to claim 9, wherein the one or more processors are configured to control the imaging unit I.

14. The system according to claim 1, further comprising one or more clamping mechanisms capable of clamping the disposable plate between the first and the second temperature-controlled plates and between at least one of the temperature-controlled plates and the clamping frame or plate.

15. The system according to claim 1, wherein the disposable plate is made of white thermoplastic polymer.

16. A method for use of the system according to claim 1, the method comprising:
loading a reaction mixture in the wells of a disposable plate and sealing the upper side of the disposable plate with a sealing foil; and
introducing the sealed disposable plate in the system, transporting and positioning it in one of the tempering units TUx in accordance with the prescribed protocol.

17. The method according to claim 16, further comprising transporting to and positioning the disposable plate in imaging unit I and capturing an image of at least one well of the disposable plate.

18. The method according to claim 16, wherein one tempering unit TUx is used per prescribed reaction temperature of the reaction protocol and at least one of the temperature-controlled plates in the tempering unit TUx is set at one of the prescribed reaction temperatures.

19. The method according to one claim 18, wherein tempering unit TUx comprises a second temperature-controlled plate and the temperature of the second temperature-controlled plates is set at a slightly higher temperature than a highest prescribed reaction temperature.

20. The method according to claim 16, wherein the chemical, biochemical or molecular biological reactions are polymerase chain reaction, real-time polymerase chain reaction or thermal shift assay.

21. The method according to claim 16, wherein the method is used for high throughput reactions or assay.


SEQUENCE LISTING (7 sequences)

SEQ ID NO: 1 - DNA, 21 bp, synthetic construct
  source 1..21: mol_type = other DNA
gcaccagtcagaccgatatgt

SEQ ID NO: 2 - DNA, 20 bp, synthetic construct
  source 1..20: mol_type = other DNA
accctgttgtcaatgcctct

SEQ ID NO: 3 - DNA, 18 bp, synthetic construct
  source 1..18: mol_type = other DNA
gtcccgctcctaaggcag

SEQ ID NO: 4 - DNA, 20 bp, synthetic construct
  source 1..20: mol_type = other DNA
gttgatcgactccagcgact

SEQ ID NO: 5 - DNA, 25 bp, synthetic construct
  source 1..25: mol_type = other DNA
aattaagcgtaactggcggaaaccc

SEQ ID NO: 6 - DNA, 25 bp, synthetic construct
  source 1..25: mol_type = other DNA
agatggtggccgcaaagaagacgaa

SEQ ID NO: 7 - RNA, 40 nt, synthetic construct
  source 1..40: mol_type = other RNA
cttgactggcgtcattcaggagctggatggcgtgggacat